(19) United States
(12) Patent Application Publication
(10) Pub. No.: US 2015/0005553 A1
(43) Pub. Date: Jan. 1, 2015

(54) Dual Riser Vortex Separation System

(71) Applicant: UOP LLC, Des Plaines, IL (US)

(72) Inventors: Paolo Palmas, Des Plaines, IL (US); Paul S. Nishimura, Arlington Heights, IL (US)

(73) Assignee: UOP LLC, Des Plaines, IL (US)

(21) Appl. No.: 13/927,422

(22) Filed: Jun. 26, 2013

(51) Int. Cl. B01D 45/12; C07C 7/00

(52) U.S. Cl. CPC B01D 45/12; C07C 7/00
USPC 585/300; 422/141; 422/255

(57) ABSTRACT

Vortex separation technology quickly and efficiently separates vapor from catalyst from two or more risers, in a singular separation vessel, controlling residence time and improving product conversion. One riser enters concentrically through the reactor vessel, then through the center of the separation vessel, ending in horizontal swirl arms. The second and any additional risers run external to the reactor vessel. The external risers transition to a 90° elbow and tangentially enter the reactor vessel, and then the separation vessel.

### CROSS-REFERENCES TO RELATED APPLICATIONS

Not Applicable.

### STATEMENT REGARDING FEDERALLY SPONSORED RESEARCH

Not Applicable.

### BACKGROUND OF THE INVENTION

1. Field of the Invention

This invention relates generally to an apparatus and a process for the separation of solid particles from gases. More specifically, this invention relates to the a singular separation apparatus for the recovery of particulate catalyst materials from gaseous materials derived from two distinct fluid catalytic cracking (FCC) processes.

2.Description of the Related Art

Cyclonic methods for the separation of solids from gases are well known and commonly used. A particularly well known application of such methods is in the hydrocarbon processing industry where particulate catalysts contact gaseous reactants to effect chemical conversion of the gas stream components or physical changes in the particles undergoing contact with the gas stream.

The FCC process presents a familiar example of a process that uses gas streams to contact a finely divided stream of catalyst particles and effects contact between the gas and the particles. The FCC processes, as well as separation devices used therein are described in U.S. Pat. Nos. 4,701,307 and 4,792,437.

Efficient separation of particulate catalyst from product vapors is very important in an FCC process. Particulate catalyst that is not effectively separated from product vapors in the FCC unit must be separated downstream either by filtration methods or additional separation devices that multiplicate separation devices utilized in the FCC unit. Additionally, catalyst that is not recovered from the FCC process represents a two-fold loss. The catalyst must be replaced, representing a material cost, and catalyst lost may cause erosion to downstream equipment. Severe erosion may cause equipment failure and subsequent lost production time. Accordingly, methods of efficiently separating particulate catalyst materials from gaseous fluids in an FCC process are of great utility.

In the FCC process, gaseous fluids are separated from particulate catalyst solids as they are discharged from a reaction conduit. The most common method of separating particulate solids from a gas stream uses centripetal separation. Centripetal separators are well known and operate by imparting a tangential velocity to gases containing entrained solid particles that forces the heavier solids particles outwardly away from the lighter gases for upward withdrawal of gases and downward collection of solids.

U.S. Pat. Nos. 4,397,738 and 4,482,451 disclose an arrangement for initial quick centripetal separation that tangentially discharges a mixture of gases and solid particles from a central reaction conduit into a containment vessel. The containment vessel has a relatively large diameter and generally provides a first separation of solids from gases. In these arrangements, the initial stage of separation is typically followed by a second more compete separation of solids from gases in a traditional cyclone device.

Another method of obtaining this initial quick separation on discharge from the reaction conduit is disclosed in U.S. Pat. No. 5,584,985. This patent discloses the contacting of feed and catalyst particles in a riser conduit. The exit from the riser conduit comprises an arcuate, tubular swirl arm which imparts a swirling, helical motion to the gases and particulate catalyst as they are discharged from the riser conduit into a separation vessel. The swirling, helical motion of the materials in the separation vessel effect an initial separation of the particulate catalyst from the gases. The swirl motion of the mixture continues while it rises up the gas recovery conduit. At the end of the gas recovery conduit, the mixture is drawn into cyclones to effect further separation of the particulate catalyst from the gases. This arrangement is known as the UOP's (VSS^{SM}) technology.

Cyclones for separating particulate material from gaseous materials are well known to those skilled in the art of FCC processing. Cyclones usually comprise an inlet that is tangential to the outside of a cylindrical vessel that forms an outer wall of the cyclone. In the operation of an FCC cyclone, the entry and the inner surface of the outer wall cooperate to create a spiral flow path of the gaseous materials and catalyst that establishes a vortex in the cyclone. The centripetal acceleration associated with an exterior of the vortex causes catalyst particles to migrate towards the outside of the barrel while the gaseous materials enter an interior of the vortex for eventual discharge through an upper outlet. The heavier catalyst particles accumulate on the side wall of the cyclone barrel and eventually drop to the bottom of the cyclone and out via an outlet and a dipleg conduit for recycle through the FCC apparatus. Cyclone arrangements and modifications thereto are generally disclosed in U.S. Pat. Nos. 4,670,410 and 2,535,140.

U.S. Pat. No. 4,956,091 discloses a separator comprising a swirl chamber that imparts a swirl motion to a mixture of gases and solids in an angular direction. The mixture then enters a swirl tube through swirl veins which intensify the swirl motion of the mixture in the same angular direction to effect separation between the solids and gases. This same principle has been followed in separation systems that are used in conjunction with cyclones. The angular direction of the swirl motion induced by the VSS^{SM} device has the same angular direction as the swirl motion induced by the cyclones. It was, perhaps, thought that consistency between the swirl motion in the VSS^{SM} device and the cyclones will operate to intensify the swirl motion in the cyclone and thereby effect greater separation.

It has been recognized in the art that there is a need for a process or apparatus to accommodate the effluent of two or more reactors or other sources of solid particles mixed with gases in order to effect a separation. One approach is to have a distinct separation process and apparatus for each mixture stream. However, this requires a large capital investment which is not desirable. Therefore, what is needed is a single separation process and apparatus that can accommodate multiple distinct streams of mixed gases and solid particles.

### SUMMARY OF THE INVENTION

In one embodiment, the present invention is a process for the fluidized catalytic cracking of a hydrocarbon feedstock. The method includes the steps of (a) passing a hydrocarbon feedstock and solid catalyst particles into a first riser to produce a first mixture of solid particles and gaseous fluids, the first riser residing within a first reactor vessel; (b) passing a hydrocarbon feedstock and solid catalyst particles into a second riser to produce a second mixture of solid particles and gaseous fluids; (c) passing the first mixture of solid particles and gaseous fluids from the first riser into a separation vessel, wherein the first riser occupies a central portion of the separation vessel and the separation vessel is located within the first reactor vessel; and (d) passing the second mixture of the solid particles and gaseous fluids from the second riser into the separation vessel, wherein the second riser intersects a wall of the separation vessel.

In one aspect, the process further includes tangentially discharging the first mixture from the first riser into the separation vessel through a first discharge opening.

In another aspect, the first mixture and second mixture flow in a circumferential path defined by the side wall of the separation vessel.

In another aspect, the process includes tangentially discharging the second mixture from the second riser into the separation vessel through a second discharge opening.

In another aspect, the first mixture and second mixture flow in a circumferential path defined by the side wall of the separation vessel.

In another aspect, the first mixture and the second mixture flow are rotated or otherwise turned in a substantially horizontal plane in the separation vessel.

In another aspect, the first mixture and the second mixture flow are rotated or otherwise turned in a substantially vertical plane in the separation vessel.

In another aspect, the gaseous fluids from the separation vessel are separated in a cyclone separator, and catalyst particles from the cyclone are passed to a stripping zone.

In a second embodiment, the present invention provides a process for the fluidized catalytic cracking of a hydrocarbon feedstock. The process includes the steps of (a) passing a hydrocarbon feedstock and solid catalyst particles into a first riser to produce a first mixture of solid particles and gaseous fluids, the first riser residing within a first reactor vessel; (b) passing a hydrocarbon feedstock and solid catalyst particles into a plurality of additional risers to produce a mixture of solid particles and gaseous fluids associated with each additional riser; (c) passing the first mixture of solid particles and gaseous fluids from the first riser into a separation vessel, wherein the first riser occupies a central portion of the separation vessel and the separation vessel is located within the first reactor vessel; and (d) passing the mixture of solid particles and gaseous fluids associated with each additional riser into the separation vessel, wherein each of the plurality of additional risers intersects a side wall of the separation vessel.

In one aspect, the process further includes tangentially discharging the first mixture from the first riser into the separation vessel through a first discharge opening.

In another aspect, the process further includes tangentially discharging the mixture of solid particles and gaseous fluids associated with each additional riser into the separation vessel through a discharge opening of each additional riser.

In another aspect, the first mixture and the mixture of solid particles and gaseous fluids associated with each additional riser flow in a circumferential path defined by the side wall of the separation vessel.

In another aspect, the first mixture and the mixture of solid particles and gaseous fluids associated with each additional riser flow are rotated or otherwise turned in a substantially horizontal plane in the separation vessel.

In another aspect, the first mixture and the mixture of solid particles and gaseous fluids associated with each additional riser flow are rotated or otherwise turned in a substantially vertical plane in the separation vessel.

In another aspect, the gaseous fluids from the separation vessel are separated in a cyclone separator, and catalyst particles from the cyclone are passed to a stripping zone.

In a third embodiment, the invention provides an apparatus for separating solid particles from a gaseous fluid. The apparatus includes a first riser conduit comprising a first discharge opening, the first riser conduit residing within a first reactor vessel, a second riser conduit comprising a second discharge opening, and a separation vessel located within the first reactor vessel, the first discharge opening and the second discharge opening being in fluid communication with the separation vessel. The first conduit occupies a central portion of the separation vessel and the second discharge opening is positioned in a side wall of the separation vessel.

In one aspect, the first riser conduit further comprises at least one additional discharge opening.

In another aspect, the second riser conduit further comprises at least one additional discharge opening.

In another aspect., the first discharge opening is oriented to discharge a first mixture of solid particles and gaseous fluid tangential to the side wall of the separation vessel. In another aspect, the second discharge opening is oriented to discharge a second mixture of solid particles and gaseous fluid tangential to the side wall of the separation vessel.

It is therefore an advantage of the invention to use a single disengaging vessel when an FCC reactor includes two or more separate risers.

In one example embodiment, a dual riser vortex separation system includes a vertical chamber, residing within an FCC reactor vessel downstream of the risers, and upstream of the reactor cyclones. The vapor and catalyst in both risers are flowing vertically, in a well mixed fluidized state. One riser (primary riser) enters concentrically through the reactor vessel, then through the center of the chamber, ending in horizontal swirl arms. The swirl arms branch off of the riser, forcing the stream of catalyst and vapor tangentially against the sides of vessel. The second riser (and other risers if any) runs external to the reactor vessel. It transitions to a 90° elbow, from which it tangentially enters the reactor vessel, and then the chamber, below the arms of the first riser. The second riser also directs its material tangentially against the wall of the chamber, following the same direction of swirl as the material from the first riser. As catalyst and vapor swirl along the chamber wall, they separate; vapor and some catalyst enter a single stage of cyclones above the chamber; the rest of the catalyst is sent to the spent catalyst stripper below the chamber.

These and other features, aspects, and advantages of the present invention will become better understood upon consideration of the following detailed description, drawings and appended claims.

### BRIEF DESCRIPTION OF THE DRAWINGS

FIG. 1 is a sectional elevational view of an FCC reactor vessel and a second, distinct FCC reactor riser schematically showing a separation vessel arranged in accordance with this invention.

FIG. 2 is a sectional elevational view of an FCC reactor vessel and a number of additional, distinct FCC reactor risers, schematically showing a separation vessel arranged in accordance with this invention.

FIG. 3 is a cross-sectional view of the separation vessel of FIG. 2 taken along the line 3-3 of FIG. 2.

Like reference numerals will be used to refer to like parts from Figure to Figure in the following description of the drawings.

### DETAILED DESCRIPTION OF THE INVENTION

A general understanding of the process and apparatus of this invention can be obtained by reference to the Figures. The Figures have been simplified by the deletion of a large number of apparatuses customarily employed in a process of this nature, such as vessel internals, temperature and pressure controls systems, flow control valves, recycle pumps, etc. which are not specifically required to illustrate the performance of the invention. Furthermore, the illustration of the process of this invention in the embodiment of a specific drawing is not intended to limit the invention to specific embodiments set out herein. Lastly, although a process for recovery of catalyst particles from FCC effluent gases is illustrated by way of an example, other gas-solids recovery schemes are contemplated.

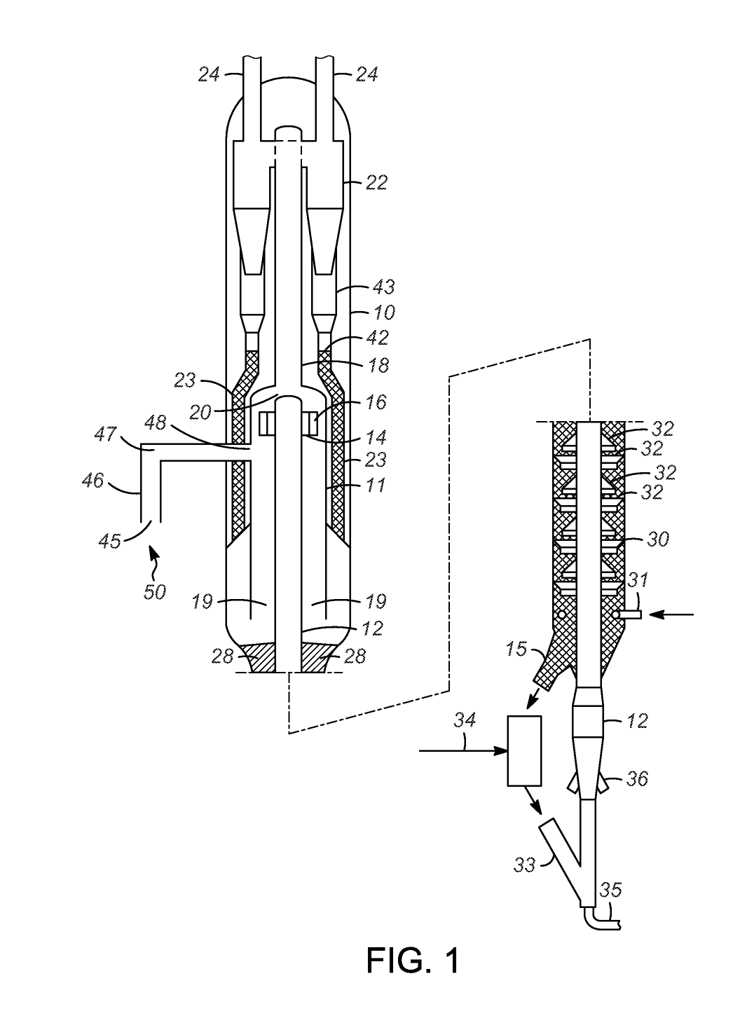

Looking then at FIG. 1, the schematic illustration depicts a separation arrangement in a reactor vessel 10. A central conduit in the form of a reactor riser 12 extends upwardly from a lower portion of the reactor vessel 10 in an FCC arrangement. The central conduit or riser preferably has a vertical orientation within the reactor vessel 10 and may extend upwardly from the bottom of the reactor vessel or downwardly from the top of the reactor vessel. Riser 12 terminates in an upper portion of a separation vessel 11 with a curved conduit in the form of an arm 14. Arm 14 discharges a mixture of gaseous fluids and solid particles comprising catalyst.

Tangential discharge of gases and catalyst from a discharge opening 16 produces a swirling helical pattern about the interior of separation vessel 11 below the discharge opening 16. Centripetal acceleration associated with the helical motion forces the heavier catalyst particles to the outer portions of separation vessel 11. Catalyst from discharge openings 16 collects in the bottom of separation vessel 11 to form a dense catalyst bed 28.

A second, distinct reactor riser 50 (and any additional reactor risers, if any) runs external to the reactor vessel 10. A second stream of gases and catalyst pass through a conduit 45 in the upper end 46 of the second reactor riser 50. The upper end 46 transitions to a 90° elbow 47 such that the upper end 46 tangentially enters the reactor vessel 10, and then the interior of the separation vessel 11, below the arm 14. In other embodiments of the present invention the elbow 47 may be exchanged for an alternative connector such a T-type connector or an elbow with a more acute or more obtuse angle. Tangential discharge of gases and catalyst from a second discharge opening 48 produces a swirling helical pattern about the interior of separation vessel 11 below the second discharge opening 48. Generally, the cross-sectional area of the second discharge opening 48 may be similar to that of the upper end 46 of the reactor riser 50, where the upper end 46 of the reactor riser 50 is about 0.3 meters (1 foot) to about 2.74 meters (9 feet) in diameter. Preferably, the upper end of the reactor riser 50 may be about 0.91 meters (3 feet) to about 2.1 meters (7 feet) in diameter. The swirling helical pattern followed by the gases and catalyst discharged from the discharge opening 48 follows the same direction of swirl as the material from the first riser. Centripetal acceleration associated with the helical motion forces the heavier catalyst particles to the outer portions of separation vessel 11. Catalyst from discharge opening 48 collects in the bottom of separation vessel 11 to form a dense catalyst bed 28.

The total gases from all of the reactor risers, having a lower density than the solids, more easily change direction and begin an upward spiral with the gases ultimately traveling into a gas recovery conduit 18 having an inlet 20. In one form of the invention (not depicted by FIG. 1), inlet 20 is located below the discharge opening 16. The gases that enter gas recovery conduit 18 through inlet 20 will usually contain a light loading of catalyst particles. Inlet 20 recovers gases from the discharge conduit as well as stripping gases which are hereinafter described. The loading of catalyst particles in the gases entering conduit 18 are usually less than 16 grams/liter (1 lb/ft³) and typically less than 1.6 grams/liter (0.1 lb/ft³).

Gas recovery conduit 18 passes the separated gases into cyclones 22 that effect a further removal of particulate material from the gases in the gas recovery conduit. Cyclones 22 operate as conventional direct connected cyclones in a conventional manner with the tangential entry of the gases creating a swirling action inside the cyclones to establish the well known inner and outer vortexes that separate catalyst from gases. A product stream, relatively free of catalyst particles, exits the reactor vessel 10 through outlets 24.

Catalyst recovered by cyclones 22 exits the bottom of the cyclone through dipleg conduits 23 and passes through a lower portion of the reactor vessel 10 where it collects with catalyst that exits separation vessel 11 through an open bottom 19 to form a dense catalyst bed 28. Catalyst from catalyst bed 28 passes downwardly through a stripping vessel 30. A stripping fluid, typically steam enters a lower portion of stripping vessel 30 through a distributor 31. Countercurrent contact of the catalyst with the stripping fluid through a series of stripping baffles 32 displaces product gases from the catalyst as it continues downwardly through the stripping vessel.

Stripped catalyst from stripping vessel 30 passes through a conduit 15 to a catalyst regenerator 34 that rejuvenates the catalyst by contact with an oxygen-containing gas. High temperature contact of the oxygen-containing gas with the catalyst oxidizes coke deposits from the surface of the catalyst. Following regeneration catalyst particles enter the bottom of reactor riser 12 through a conduit 33 where a fluidizing gas from a conduit 35 pneumatically conveys the catalyst particles upwardly through the riser. As the mixture of catalyst and conveying gas continues up the riser, nozzles 36 inject feed into the catalyst, the contact of which vaporizes the feed to provide additional gases that exit through discharge opening 16 in the manner previously described.

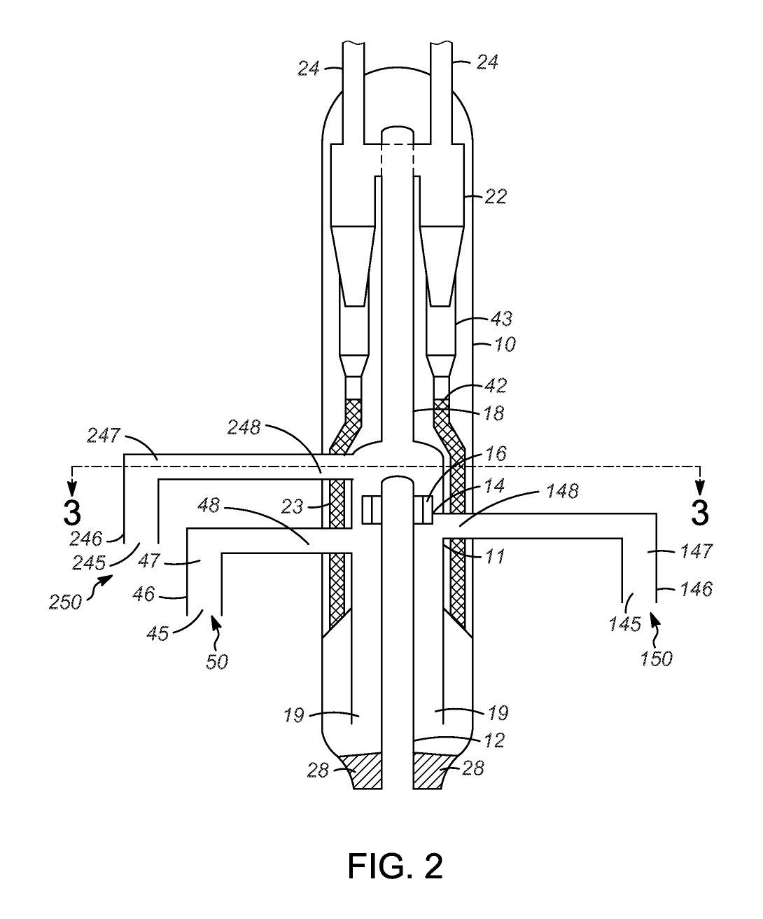

FIG. 2 shows a sectional elevation of an FCC reactor vessel analogous to the FCC reactor shown in FIG. 1, wherein more than one additional, distinct FCC reactor riser is shown in accordance with the present invention. In FIG. 2, three distinct reactor risers 50, 150, 250 run external to the reactor vessel 10, although the use of more or less reactor risers are anticipated. The reactor riser 50 runs external to the reactor vessel 10. A second stream of gases and catalyst pass through the conduit 45 in the upper end 46 of the second reactor riser 50. The upper end 46 transitions to a 90° elbow 47 such that the upper end 46 tangentially enters the reactor vessel 10, and then the interior of the separation vessel 11, below the arm 14.

Tangential discharge of gases and catalyst from the second discharge opening 48 produces a swirling helical pattern about the interior of separation vessel 11 below the second discharge opening 48. The reactor riser 150 runs external to the reactor vessel 10. A third stream of gases and catalyst pass through a conduit 145 in the upper end 146 of the third reactor riser 150. The upper end 146 transitions to a 90° elbow 147 such that the upper end 146 tangentially enters the reactor vessel 10, and then the interior of the separation vessel 11, below the arm 14. Tangential discharge of gases and catalyst from a third discharge opening 148 produces a swirling helical pattern about the interior of separation vessel 11 below the third discharge opening 148. The reactor riser 250 runs external to the reactor vessel 10. A fourth stream of gases and catalyst pass through a conduit 245 in the upper end 246 of the fourth reactor riser 250. The upper end 246 transitions to a 90° elbow 247 such that the upper end 246 tangentially enters the reactor vessel 10, and then the interior of the separation vessel 11, above the arm 14. Tangential discharge of gases and catalyst from a fourth discharge opening 248 produces a swirling helical pattern about the interior of separation vessel 11 below the fourth discharge opening 248. The elbows 47, 147, 247 could be configured to form an angle in the range of 45° to 135°, in the range of 60° to 120°, or in the range of 75° to 105°, to the upper ends 46, 146, 246 of the risers 50, 150, 250, respectively.

Tangential discharge of gases and catalyst from the additional discharge openings 48, 148, 248 produces a swirling helical pattern about the interior of separation vessel 11. The swirling helical pattern followed by the gases and catalyst discharged from the openings 48, 148, 248 follows the same direction of swirl as the material from the first riser. Centripetal acceleration associated with the helical motion forces the heavier catalyst particles to the outer portions of separation vessel 11. Catalyst from the discharge openings 48, 148, 248 collects in the bottom of separation vessel 11 to form a dense catalyst bed 28.

In FIG. 1, the discharge opening 48 is positioned below the discharge opening 16 of the arm 14 of the first, interior reactor riser 12. As seen in FIG. 2, the discharge openings 48, 148, 248 may be positioned within the separation vessel 11 in a number of different configurations. For example, a discharge opening 48 may be positioned above the discharge opening 16 of the arm 14 of the first, interior reactor riser 12. Alternatively, the discharge opening 148 may be positioned at substantially the same level as the discharge opening 16 of the arm 14 of the first, interior reactor riser 12. Alternatively, the discharge opening 148 may be positioned with any horizontal overlap with the discharge opening 16 of the arm 14 of the first, interior reactor riser 12. Alternatively, the discharge opening 248 may be positioned above the discharge opening 16 of the arm 14 of the first, interior reactor riser 12.

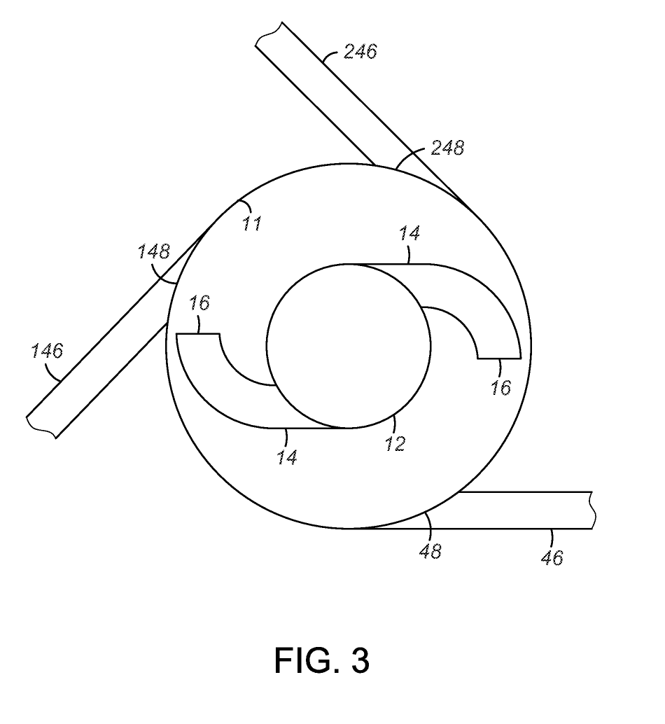

Turning now to FIG. 3, a cross-sectional view is shown of the separation vessel 11 taken along the line 3-3 of FIG. 2. In the depicted embodiment of the present invention, two arms 14 with first discharge openings 16 extend radially outward from the terminal end of the first riser 12. The upper ends 46 of the one or more additional reactor risers 50 have second discharge openings 48 where the upper ends 46 tangentially enters the separation vessel 11. Tangential discharge of gases and catalyst from the first discharge opening 16 and second discharge openings 48 produces a swirling helical pattern about the interior of separation vessel 11 below the discharge opening 16.

FIGS. 1-3 depict one preferred embodiment of the present invention in which gases and catalyst entering the separation vessel 11 through discharge openings 16 and 48 are rotated or otherwise turned in a substantially horizontal plane in the separation vessel 11. However, alternative embodiments of the present invention are envisioned in which the gases and catalyst are rotated or otherwise turned in a substantially vertical plane in the separation vessel 11. Separation methods that may be compatible with the present invention for effecting a rotation in the vertical plane are disclosed in U.S. Pat. Nos. 5,837,129 (the '129 patent) and 7,429,363 (the '363 patent). In the '129 patent, the use of one or more semi-circular separating areas is described. Gases and catalyst particles are passed directly from a reactor riser to the separating areas, which rotate the gases and catalyst in a substantially vertical plane in order to effect a separation of the gases from the catalyst particles. Similarly, the '363 patent describes a semicircular portion of a separation device positioned above the reactor riser which is adapted to rotate a mixture of gases and catalyst particles in a vertical plane.

Although the invention has been described in considerable detail with reference to certain embodiments, one skilled in the art will appreciate that the present invention can be practiced by other than the described embodiments, which have been presented for purposes of illustration and not of limitation. Therefore, the scope of the appended claims should not be limited to the description of the embodiments contained herein.

What is claimed is:

1. A process for the fluidized catalytic cracking of a hydrocarbon feedstock, the process comprising:
passing a hydrocarbon feedstock and solid catalyst particles into a first riser to produce a first mixture of solid particles and gaseous fluids, the first riser residing within a first reactor vessel;
passing a hydrocarbon feedstock and solid catalyst particles into a second riser to produce a second mixture of solid particles and gaseous fluids;
passing the first mixture of solid particles and gaseous fluids from the first riser into a separation vessel, wherein the first riser occupies a central portion of the separation vessel and the separation vessel is located within the first reactor vessel; and
passing the second mixture of the solid particles and gaseous fluids from the second riser into the separation vessel, wherein the second riser intersects a wall of the separation vessel.

2. The process of claim 1, further comprising tangentially discharging the first mixture from the first riser into the separation vessel through a first discharge opening.

3. The process of claim 2, wherein the first mixture and second mixture flow in a circumferential path defined by the side wall of the separation vessel.

4. The process of claim 1, further comprising tangentially discharging the second mixture from the second riser into the separation vessel through a second discharge opening.

5. The process of claim 4, wherein the first mixture and second mixture flow in a circumferential path defined by the side wall of the separation vessel.

6. The process of claim 4, wherein the first mixture and the second mixture flow are rotated or otherwise turned in a substantially horizontal plane in the separation vessel.

7. The process of claim 4, wherein the first mixture and the second mixture flow are rotated or otherwise turned in a substantially vertical plane in the separation vessel.

8. The process of claim 1 wherein the gaseous fluids from the separation vessel are separated in a cyclone separator, and catalyst particles from the cyclone are passed to a stripping zone.

9. A process for the fluidized catalytic cracking of a hydrocarbon feedstock, the process comprising:
passing a hydrocarbon feedstock and solid catalyst particles into a first riser to produce a first mixture of solid particles and gaseous fluids, the first riser residing within a first reactor vessel;
passing a hydrocarbon feedstock and solid catalyst particles into a plurality of additional risers to produce a mixture of solid particles and gaseous fluids associated with each additional riser;
passing the first mixture of solid particles and gaseous fluids from the first riser into a separation vessel, wherein the first riser occupies a central portion of the separation vessel and the separation vessel is located within the first reactor vessel; and
passing the mixture of solid particles and gaseous fluids associated with each additional riser into the separation vessel, wherein each of the plurality of additional risers intersects a side wall of the separation vessel.

10. The process of claim 9, further comprising:
tangentially discharging the first mixture from the first riser into the separation vessel through a first discharge opening.

11. The process of claim 9, further comprising:
tangentially discharging the mixture of solid particles and gaseous fluids associated with each additional riser into the separation vessel through a discharge opening of each additional riser.

12. The process of claim 11, wherein the first mixture and the mixture of solid particles and gaseous fluids associated with each additional riser flow in a circumferential path defined by the side wall of the separation vessel.

13. The process of claim 12, wherein the first mixture and the mixture of solid particles and gaseous fluids associated with each additional riser flow are rotated or otherwise turned in a substantially horizontal plane in the separation vessel.

14. The process of claim 12, wherein the first mixture and the mixture of solid particles and gaseous fluids associated with each additional riser flow are rotated or otherwise turned in a substantially vertical plane in the separation vessel.

15. The process of claim 9 wherein the gaseous fluids from the separation vessel are separated in a cyclone separator, and catalyst particles from the cyclone are passed to a stripping zone.

16. An apparatus for separating solid particles from a gaseous fluid, the apparatus comprising:
a first riser conduit comprising a first discharge opening, the first riser conduit residing within a first reactor vessel;
a second riser conduit comprising a second discharge opening; and
a separation vessel located within the first reactor vessel, the first discharge opening and the second discharge opening being in fluid communication with the separation vessel,
wherein the first conduit occupies a central portion of the separation vessel and the second discharge opening is positioned in a side wall of the separation vessel.

17. The apparatus of claim 16, wherein the first riser conduit further comprises at least one additional discharge opening.

18. The apparatus of claim 16, wherein the second riser conduit further comprises at least one additional discharge opening.

19. The apparatus of claim 16, wherein the first discharge opening is oriented to discharge a first mixture of solid particles and gaseous fluid tangential to the side wall of the separation vessel.

20. An apparatus for separating solid particles from a gaseous fluid, the apparatus comprising:
a first riser conduit comprising a first discharge opening;
a second riser conduit comprising a second discharge opening; and
a separation vessel located within the first reactor vessel, the first discharge opening and the second discharge opening being in fluid communication with the separation vessel,
wherein the first discharge opening is positioned in a side wall of the separation vessel, and the second discharge opening is positioned in the side wall of the separation vessel.
